(19) United States
(12) Patent Application Publication
(10) Pub. No.: US 2016/0114002 A1
(43) Pub. Date: Apr. 28, 2016

(54) COMPOSITIONS COMPRISING PLANT PROTEINS AND METHODS FOR PREVENTION OF METABOLIC AND CARDIOVASCULAR PATHOLOGIES IN PATIENT WITH CARDIOMETABOLIC RISK, INCLUDING HYPERGLYCEMIA

(71) Applicant: International Nutrition Research Company, Luxembourg (FR)

(72) Inventor: Claude Vincent, Bordeaux (FR)

(21) Appl. No.: 14/549,709

(22) Filed: Nov. 21, 2014

(30) Foreign Application Priority Data
Oct. 22, 2014 (FR) 14 60157

(51) Int. Cl. A61K 38/38; A23L 1/303; A23L 1/304; A23L 2/52; A23L 2/66; A23L 1/302; A61K 38/16; A61K 38/17; A61K 31/202; A61K 31/405; A61K 31/185; A61K 31/121; A61K 47/10; A61K 47/24; A61K 47/22; A61K 47/02; A61K 31/198; A23L 1/305

(52) U.S. Cl. CPC A61K 38/38; A23L 1/3056; A23L 1/3051; A23L 1/303; A23L 1/304; A23L 2/52; A23L 2/66; A23L 1/302; A61K 38/168; A61K 38/1709; A61K 31/202; A61K 31/405; A61K 31/185; A61K 31/121; A61K 47/10; A61K 47/24; A61K 47/22; A61K 47/02; A61K 31/198; A23V 2002/00

(57) ABSTRACT

A composition for oral administration in humans comprising at least one plant protein chosen from pea proteins, rice proteins or a mixture thereof, and:
at least one protein of animal origin chosen from calcium caseinate, lactoserum proteins, or a mixture thereof, and/or
at least one free amino acid chosen from leucine, glutamine, isoleucine, valine, tryptophan or plant 5HTP, lysine, threonine, arginine and taurine, or any mixture thereof, and/or
at least one micronutrient chosen from vitamin D, vitamin B6, vitamin B9, vitamin E, milk calcium, marine magnesium, zinc and chromium, or any mixture thereof, and/or
at least one anti-inflammatory and/or antioxidant compound of plant origin chosen from ALA omega-3 fatty acid, purified EPA, protectin in PD1 or PDX form, and curcumin, or any mixture thereof. Methods with these compositions for preventing cardiometabolic risk are also provided.

### CROSS-REFERENCE TO RELATED APPLICATIONS

This application claims its priority to French patent application FR 14 60157 filed on Oct. 22, 2014, the entire disclosure of which is incorporated herein by reference.

### FIELD OF THE INVENTION

The present invention relates to compositions with plant proteins and their use to prevent cardiometabolic risk.

### BACKGROUND

Cardiometabolic risk groups together several risk factors that may lead, over a longer or shorter term, to metabolic pathologies such as type 2 diabetes and/or cardiovascular disease. These risk factors are, in particular, increased glycaemia dyslipidemia, high blood pressure, excess abdominal fat.

A patient with cardiometabolic risk must have at least three of these factors, some combinations being more dangerous than others. It has in particular been recognized that in combinations where hyperglycemia is present, a risk is doubled with respect to other factors. It is also known that the consequences are different depending on the factors involved:
for metabolic pathologies, the main risks are: hyperglycemia, significant waist circumference, high blood pressure, low HDL cholesterol, hypertriglyceridemia, high visceral body fat mass, chronic low-grade inflammation and insulin resistance;
for type 2 diabetes or prediabetes specifically, the main risks are: hyperglycemia, abnormal insulinemia, high HbA1c level and/or abnormal evaluation of the HOMA IR, HOMA B and/or HOMA S homeostatic model and excessive weight;
for cardiovascular pathologies, the main risks are: hyperglycemia, high blood pressure, dyslipidemia, high weight, tobacco use and/or family history.

Clinically, the major factors in terms of morbidity/mortality are hyperglycemia, significant waist girth involving a significant visceral body fat mass, hypertension, low HDL cholesterol level and high triglyceride level.

In terms of the physiopathology of the cardiometabolism, the main cause is the presence of excessive visceral fat due to oxidative stress, which causes inflammation (in particular measured using the PAI-1 level, prothrombotic marker, and secondarily by more general markers such as TNα, IL6 or CRP us) and insulin resistance (determined by abnormal insulinemia and/or an abnormal evaluation of the HOMA IR, HOMA B and/or HOMA S homeostatic model).

Until recently, the existing solutions to avoid cardiometabolic risk were limited.

None of the many diets proposed since the appearance of cardiometabolic risk in the 1980s, whether balanced, hyperproteinated, hyperglucidic, hyperlipidic, very restrictive or unbalanced, have resolved the specific deficiency problems and pathological particularities of this syndrome.

Furthermore, the proposed drugs work on only one of the consequences of the metabolic syndrome, such as hyperglycemia or hypertension or hypertriglyceridemia or hypercholesterolemia, but not the entire metabolic syndrome. The pollypill proposed as prevention for cardiometabolic risk was rejected by all of the Drug Agencies.

More recently, effective solutions have been proposed, in particular the composition described in application FR2981544. In clinical trials, this composition proved to be particularly suitable for people with high waist girth and a high triglyceride level.

There is therefore a need for a composition more suited to people with a metabolic syndrome whose essential risk factor is hyperglycemia.

### SUMMARY OF THE INVENTION

The aim of the present invention is to meet this need and propose a composition able to prevent cardiometabolic risk, and therefore metabolic and/or cardiovascular pathologies resulting therefrom, by acting simultaneously on:
Clinical Risk Factors: glycaemia, waist circumference, blood pressure, HDL cholesterol, LDL cholesterol, total cholesterol and triglycerides, and
Vectors of the Physiopathology of Visceral Fat: inflammation (PAI 1) and insulin resistance (insulinemia, HOMA IR, HOMA B and HOMA S).

The present invention provides a composition suitable for oral administration and comprising at least one plant protein chosen from among pea proteins, rice proteins or mixture of pea protein(s) and rice protein(s), and:
at least one protein of animal origin chosen from among calcium caseinate, lactoserum proteins, or a mixture, and/or
at least one free amino acid chosen from among leucine, glutamine, isoleucine, valine, tryptophan or plant 5HTP, lysine, threonine, arginine and taurine, or a mixture of at least two of these amino acids, and/or
at least one micronutrient chosen from among vitamin D, vitamin B6, vitamin B9, vitamin E, milk calcium, marine magnesium, zinc and chromium, or a mixture of at least two of these micronutrients, and/or
at least one anti-inflammatory and/or antioxidant compound of plant origin chosen from among ALA omega-3 fatty acid, purified EPA, protectin in PD1 or PDX form, and curcumin, or mixture of at least two of these anti-inflammatory compounds of plant origin.

Advantageously, such a composition may be used as a healthcare product, in particular as a medical nutrition product, to prevent cardiometabolic risk, in particular to prevent metabolic and/or cardiovascular pathologies associated with cardiometabolic risk. The composition according to the invention may in particular be used to act on vectors of the visceral fat physiopathology and clinical risk factors for cardiometabolic risk. The invention is particularly suitable for cardiometabolic risk for patients whose main risk factor is hyperglycemia.

### DETAILED DESCRIPTION

The invention will now be described in detail.

The invention therefore relates to a particular composition suitable for oral administration in humans comprising at least one plant protein chosen from among pea proteins, rice proteins or mixture of pea protein(s) and rice protein(s), and:
at least one protein of animal origin chosen from among calcium caseinate, lactoserum proteins, or a mixture, and optionally another animal protein, in particular a fish protein, and/or
at least one free amino acid chosen from among leucine, glutamine, isoleucine, valine, tryptophan or plant 5HTP, lysine, threonine, arginine and taurine, or a mixture of at least two of these amino acids, and/or
at least one micronutrient chosen from among vitamin D, vitamin B6, vitamin B9, vitamin E, milk calcium, marine magnesium, zinc and chromium, or a mixture of at least two of these micronutrients, and/or
at least one anti-inflammatory and/or antioxidant compound of plant origin chosen from among ALA omega-3 fatty acid, purified EPA, protectin in PD1 or PDX form, and curcumin, or mixture of at least two of these anti-inflammatory compounds of plant origin.

Preferably, the composition according to the invention does not comprise other components except excipients.

The composition therefore comprises at least plant proteins and one or more elements belonging to one of the four following groups:
the group of animal proteins,
the group of free amino acids,
the group of micronutrients,
the group of plant anti-inflammatory compounds.

The composition therefore comprises at least one plant protein chosen from among pea proteins and rice proteins, or several pea proteins, or several rice proteins, or mixture of one or more pea proteins and one or more rice proteins.

The plant proteins may be in native form (isolate or concentrate or isolate and concentrate mixture) or in hydrolyzed form (hydrolysate).

Preferably, the plant protein(s) are in the form of an isolate or concentrate.

If the plant protein is in hydrolyzed form, the hydrolysis level varies from 10 to 35%.

Still more preferably, the pea proteins(s) are present in isolate form with a high viscosity level to obtain good solubility, and that isolate must make it possible to obtain at least an 85% level of pure proteins. The rice protein(s) are preferably in rice protein isolate form with a level of at least 85% pure proteins.

The plant proteins used in the composition according to the invention have the advantage of acting on the microbiota, the intestinal barrier of the adipose tissue and the adipocyte, and they have a clinical impact on glycaemia and insulin resistance, the lipolysis, and lastly the dyslipidemia and the antithrombotic effect.

The composition may also comprise at least one animal protein chosen from among calcium caseinate and lactoserum proteins, or a mixture of at least two of those animal proteins.

The animal proteins may be in native form (isolate or concentrate or mixture of isolate and concentrate) or in hydrolyzed form (hydrolysate).

Preferably, the composition comprises at least one calcium caseinate.

If the composition comprises lactoserum proteins, it preferably comprises lactoserum in isolate, concentrate and/or hydrolysate form, or α-lactalbumin.

If the animal protein is in hydrolyzed form, the hydrolysis level preferably varies from 10 to 35%.

The animal protein(s) of the composition according to the invention act on the AMPK and PPAR energy cycle, the adipose tissue with lipolysis, the differentiation of pre-adipocytes and above all the inflammation cycle by infiltrated macrophages.

The total quantity of whole proteins (without taking any free amino acids added in the composition into account) present in a daily ration of the composition according to the invention for oral administration to humans is preferably comprised between 15 and 50 g, preferably distributed in two doses.

The proteins present in the composition according to the invention are made up of amino acids participating in the efficacy of the composition on the prevention of cardiometabolic risk, in particular:
the leucine and isoleucine, which participate in synthesis of the proteins and act on ketogenesis,
the valine and glutamine, which participate in protein synthesis,
the alanine, which acts on the energy cycle,
the lysine, tyrosine, phenylalanine, which act on ketogenesis,
the tryptophan, which acts on satiety, mood and sleep as well as ketogenesis,
the threonine, which act on the intestinal barrier,
arginine and taurine, which act on inflammation.

Preferably, the protein(s) of the composition according to the invention comprise at least the following amino acids: leucine, isoleucine, valine, glutamine, tryptophan, lysine, alanine, tyrosine, phenylalanine, threonine. Still more preferably:
leucine represents at least 7% of all of the amino acids present in the protein(s) of the composition (weight percentage relative to the total weight of the amino acids present in the protein(s) of the composition): this in particular makes it possible to act on the 3HMGCoA ketogenesis signal,
and/or tryptophan, lysine and leucine together represent between 10 and 25% of the amino acids present in the protein(s) of the composition (weight percentage relative to the total weight of the amino acids present in the protein(s) of the composition): this in particular makes it possible to act on the acetyl CoA ketogenesis signal,
and/or the branched amino acids and glutamine together represent between 20 and 40% of the amino acids present in the protein(s) of the composition (weight percentage relative to the total weight of the amino acids present in the protein(s) of the composition): this in particular makes it possible to act on muscle proteolysis,
and/or the alanine and tryptophan together represent between 3 and 5% of the amino acids present in the protein(s) of the composition (weight percentage relative to the total weight of the amino acids present in the protein(s) of the composition): this in particular makes it possible to favor the manufacture of pyruvate for the energy cycle, and to counter the tryptophan deficiency of people with abdominal obesity,
and/or the tyrosine and phenylalanine together represent between 7 and 10% of all of the amino acids present in the protein(s) of the composition (weight percentage relative to the total weight of the amino acids present in the protein(s) of the composition): this in particular makes it possible to act on the 3OH butyrate, which is a ketogenesis indicator and also acts at the cerebral level,
and/or the weight ratio of arginine to taurine is comprised between 1.2 and 2.

Thus, the proteins present in the composition according to the invention preferably act on the energy, ketogenesis and muscle proteolysis cycles.

The composition according to the invention may also comprise free amino acids. These amino acids reinforce the action of whole proteins in the energy cycle, ketogenesis, muscle proteolysis and anti-inflammatory action.

In particular, the composition according to the invention may comprise a free amino acid chosen from among leucine, threonine, glutamine, isoleucine, valine, tryptophan or plant 5HTP, lysine, arginine and taurine, or a mixture of at least two of those amino acids. According to one preferred embodiment, the composition comprises the following free amino acids: leucine, tryptophan or plant 5HTP, threonine, taurine and arginine.

The total daily leucine intake for person with abdominal obesity should be between 3 and 5 g, branched amino acids between 6.5 and 10.5 g, tryptophan between 0.4 and 0.8 g, neutral amino acids between 10 and 16 g and threonine between 1 and 2.5 g; to preserve a low body weight and maintain the energy cycle, the group of tryptophan, leucine and lysine amino acids must be between 5 and 10 g. The aim of the present invention is to cover, by the whole proteins and free amino acids, between 50 and 100% of the average quantity necessary for all of those amino acids or amino acid groups, knowing that these people have a deficiency and that a greater intake for a period limited to 12 to 24 weeks is sometimes necessary to regulate the different cycles, including the energy cycle.

The composition according to the invention may also comprise at least one micronutrient chosen from among vitamin D, vitamin B6, vitamin B9, vitamin E, milk calcium, marine magnesium, zinc and chromium, or a mixture of at least two of those micronutrients. Preferably, it comprises all of those micronutrients.

These micronutrients make it possible to offset deficiencies in people with cardiometabolic risk, generally with abdominal obesity.

Lastly, the composition also comprises at least one anti-inflammatory and/or antioxidant compound of plant origin chosen from among ALA omega-3 fatty acid, purified EPA, protectin in PD1 or PDX form, and curcumin, or a mixture of at least two of these anti-inflammatory compounds of plant origin. Preferably, it comprises ALA and curcumin.

ALA is the plant form of the omega-3 precursor for EPA, whose essential action is anti-inflammatory, in particular on the endothelial barrier. The composition may also contain purified EPA. This molecule allows better protection at the intestinal barrier to prevent LPS from passing that barrier.

Curcumin has a local anti-inflammatory effect on the intestinal barrier as well, which is combined with that of the glutamine present in the composition.

The composition according to the invention may be obtained using a method as described below:
a first mixture is obtained by mixing the components in the following order: plant proteins, animal proteins, free amino acids. The pH must be around 7 and stabilized at that level.
Addition of vitamins, minerals and anti-inflammatories to the first mixture.

This obtains a powder that can be transformed into a tablet or liquid, or used in its powder form in sachets, sticks, cans or gelcaps, for example.

The composition according to the invention may assume any form suitable for oral administration. It may in particular assume the form of powder or granules, ready-to-use beverages or bars or an extruded form, the composition being supplemented by excipients and traditional fillers known by those skilled in the art.

Preferably, it assumes the form of powder or granules packaged in a sachet to be diluted in water.

Advantageously, the various components of the composition act synergistically to affect risk factors for cardiometabolic risk.

The composition according to the invention can therefore be used as a healthcare product, in particular a medical nutrition product, to prevent cardiometabolic risk, in particular to prevent metabolic and/or cardiovascular pathologies resulting from cardiometabolic risk.

Within the meaning of the present invention, “medical nutrition product” refers to a particular healthcare product, namely a food or food supplement for therapeutic use, alone or combined with other therapies.

The composition according to the invention, when administered orally in a sufficient quantity, makes it possible to act directly on different risk factors for cardiometabolic risk, in particular:
clinical and biological risk factors: waist circumference, blood pressure, HDL cholesterol, LDL cholesterol, total cholesterol, triglycerides, glycaemia, and
vectors of the physiopathology of visceral fat: inflammation (PAI 1) and insulin resistance (insulinemia, HOMA IR, HOMA B and HOMA S).

It is particularly suitable for people with risk factors for cardiometabolic risk, with increased prediabetic glycaemia. This combination is, however, recognized as the most dangerous combination of risk factors for cardiometabolic risk.

It also has surprising efficacy on hyperlipidemia risk factors.

Furthermore, it acts very powerfully on the regulation of intestinal microbiota, in particular by increasing bacteriodetes and decreasing firmicutes. It is also able to decrease inflammation of the intestinal barrier to decrease the passage of LPS.

It also makes it possible to increase lipolysis of the adipocytes, stop differentiation of pre-adipocytes, and reduce the size of adipocytes, as well as modulate the inflammatory expression of macrophages in the tissue.

The invention therefore targets the composition as previously described for its use as an oral medical nutrition product to prevent cardiometabolic risk in human beings, in particular to prevent metabolic and/or cardiovascular pathologies associated with cardiometabolic risk, in particular type 2 diabetes and cardiovascular diseases.

It is particularly effective to prevent cardiometabolic risk with the risk of hyperglycemia as the main factor.

The composition according to the invention may be used as an oral medical nutrition product to prevent and/or combat prediabetes.

The invention also specifically relates to the use of the composition to prevent and/or combat at least one of the risk factors for cardiometabolic risk, in particular to decrease glycaemia, but also to decrease waist circumference, normalize blood pressure, increase HDL cholesterol, decrease LDL cholesterol, decrease total cholesterol, decrease triglycerides, decrease visceral body fat mass, decrease inflammation (particularly decrease PAI 1, pro-thrombotic adipocytokine) and decrease insulin resistance (in particular to regulate insulinemia, HOMA IR, HOMA B and/or HOMA S).

Lastly, the invention may also be used to regulate intestinal microbiota, in particular by increasing bacteriodetes and decreasing firmicutes.

The daily dose of the composition according to the invention (dose of mixture of active ingredient without excipients) is preferably comprised between 60 and 120 g, preferably in two doses of 30 to 60 g taken one in the morning with breakfast or at 11 a.m. with a snack, and one with a snack in the afternoon.

The composition according to the invention should preferably be accompanied by a calorie restriction comprised between 500 and 800 kcal for a resting energy expenditure (REE) of about 1,800 kcal.

The invention is illustrated through examples and trial results demonstrating the efficacy of the composition according to the invention.

### EXAMPLE 1

The composition of example 1 is made up of:

| | |
|---|---|
| Quantity for a | Quantity for |
| daily ratio | a dose |

PROTEINS

| | | |
|---|---|---|
| Peas | 10 | 5 |
| Lactoserum | 8 | 4 |
| hydrolysate DH | | |
| 13% | | |
| Lactoserum | 8 | 4 |
| isolate | | |
| Lactoserum | 8 | 4 |
| concentrate | | |
| Calcium | 16 | 8 |
| caseinate | | |

FREE AMINO ACIDS

| | | | | |
|---|---|---|---|---|
| Tryptophan | 0.5 | g | 0.25 | g |
| Taurine | 1.0 | g | 0.5 | g |

MICRONUTRIENTS

| | | | | |
|---|---|---|---|---|
| Vitamin D | 5 | μg | 2.5 | μg |
| Vitamin B6 | 1.4 | mg | 0.7 | mg |
| Vitamin B9 | 200 | μg | 100 | μg |
| Vitamin E | 12 | mg | 6 | mg |
| Milk calcium | 600 | mg | 300 | mg |
| Marine | 300 | mg | 150 | mg |
| magnesium | | | | |
| Zinc | 10 | mg | 5 | mg |
| Chromium | 40 | μg | 20 | μg |

ANTI-INFLAMMATORY

| | | | | |
|---|---|---|---|---|
| ALA (ω3) | 1000 | mg | 500 | mg |
| Curcumin | 240 | mg | 120 | mg |

Furthermore, the amino acids present in the composition (amino acids making up proteins and free amino acids) are the following:

| | Quantity for a daily | |
|---|---|---|
| | ration g | Quantity for a dose g |
| Leucine | 5.3 | 2.6 |
| Isoleucine | 2.8 | 1.4 |
| Valine | 3.0 | 1.5 |
| phenylalanine + tyrosine | 4.5 | 2.3 |
| Tryptophan | 1.4 | 0.7 |
| Neutral amino acids | 5.5 | 2.75 |
| Lysine | 4.5 | 2.3 |
| Arginine | 2.2 | 1.1 |
| TAURINE | 1.0 | 0.5 |
| Threonine | 2.3 | 1.1 |

### EXAMPLE 2

The composition of example 2 is made up of:

| | |
|---|---|
| Quantity for a | Quantity for |
| daily ration | a dose |

PROTEINS

| | | | | |
|---|---|---|---|---|
| Peas | 10 | g | 5 | g |
| Lactoserum | 12 | g | 6 | g |
| hydrolysate DH | | | | |
| 25% | | | | |
| α-lactalbumin | 12 | g | 6 | g |
| Calcium | 10 | g | 5 | g |
| caseinate | | | | |

FREE AMINO ACIDS

| | | | | |
|---|---|---|---|---|
| Tryptophan | 0.5 | g | 0.25 | g |
| Taurine | 1.0 | g | 0.5 | g |

MICRONUTRIENTS

| | | | | |
|---|---|---|---|---|
| Vitamin D | 5 | μg | 2.5 | μg |
| Vitamin B6 | 1.4 | mg | 0.7 | mg |
| Vitamin B9 | 200 | μg | 100 | μg |
| Vitamin E | 12 | mg | 6 | mg |
| Milk calcium | 600 | mg | 300 | mg |
| Marine | 300 | mg | 150 | mg |
| magnesium | | | | |
| Zinc | 10 | mg | 5 | mg |
| Chromium | 40 | μg | 20 | μg |

ANTI-INFLAMMATORY

| | | | | |
|---|---|---|---|---|
| ALA (ω3) | 1000 | mg | 500 | mg |
| Curcumin | 240 | mg | 120 | mg |

Furthermore, the amino acids present in the composition (amino acids making up proteins and free amino acids) are the following:

| | Quantity for a daily | |
|---|---|---|
| | ration g | Quantity for a dose g |
| Leucine | 4.4 | 2.2 |
| Isoleucine | 2.4 | 1.2 |
| Valine | 2.5 | 1.3 |
| phenylalanine + tyrosine | 4.0 | 2.0 |
| Tryptophan | 1.4 | 0.7 |
| Neutral amino acids | 14.2 | 7.1 |
| Lysine | 3.9 | 1.9 |
| Arginine | 1.8 | 0.9 |
| TAURINE | 1.0 | 0.5 |
| Threonine | 2.1 | 1.1 |

### EXAMPLE 3

The composition of example 3 is made up of:

| | |
|---|---|
| Quantity for a | Quantity for |
| daily ration | a dose |

PROTEINS

| | | | | |
|---|---|---|---|---|
| Peas | 9 | g | 4.5 | g |
| Calcium | 7 | g | 3.5 | g |
| caseinate | | | | |

FREE AMINO ACIDS

| | | | | |
|---|---|---|---|---|
| Leucine | 1.5 | g | 0.75 | g |
| Tryptophan | 0.5 | g | 0.25 | g |
| Taurine | 1.0 | g | 0.5 | g |
| Arginine | 0.5 | g | 0.25 | g |
| Threonine | 0.50 | g | 0.25 | g |

MICRONUTRIENTS

| | | | | |
|---|---|---|---|---|
| Vitamin D | 5 | μg | 2.5 | μg |
| Vitamin B6 | 1.4 | mg | 0.7 | mg |
| Vitamin B9 | 200 | μg | 100 | μg |
| Vitamin E | 12 | mg | 6 | mg |
| Milk calcium | 600 | mg | 300 | mg |
| Marine | 300 | mg | 150 | mg |
| magnesium | | | | |
| Zinc | 10 | mg | 5 | mg |
| Chromium | 40 | μg | 20 | μg |

ANTI-INFLAMMATORY

| | | | | |
|---|---|---|---|---|
| ALA (ω3) | 1000 | mg | 500 | mg |
| Curcumin | 240 | mg | 120 | mg |

Furthermore, the amino acids present in the composition (amino acids making up proteins and free amino acids) are the following:

| | Quantity for a daily | |
|---|---|---|
| | ration g | Quantity for a dose g |
| Leucine | 2.93 | 1.46 |
| Isoleucine | 0.83 | 0.41 |
| Valine | 0.9 | 0.45 |
| phenylalanine + tyrosine | 1.65 | 0.82 |
| Tryptophan | 0.70 | 0.36 |
| Neutral amino acids | 7.00 | 3.5 |
| Arginine | 1.6 | 0.8 |
| TAURINE | 1.0 | 0.5 |
| Threonine | 1.15 | 0.8 |

### EXAMPLE 4

The composition of example 4 is made up of:

| | |
|---|---|
| Quantity for a | Quantity for |
| daily ration | a dose |

PROTEINS

| | | | | |
|---|---|---|---|---|
| Peas | 24 | g | 12 | g |
| Calcium | 16 | g | 8 | g |
| caseinate | | | | |

FREE AMINO ACIDS

| | | | | |
|---|---|---|---|---|
| Tryptophan | 0.5 | g | 0.25 | g |

MICRONUTRIENTS

| | | | | |
|---|---|---|---|---|
| Vitamin D | 5 | μg | 2.5 | μg |
| Vitamin B6 | 1.4 | mg | 0.7 | mg |
| Vitamin B9 | 200 | μg | 100 | μg |
| Vitamin E | 12 | mg | 6 | mg |
| Milk calcium | 600 | mg | 300 | mg |
| Marine | 300 | mg | 150 | mg |
| magnesium | | | | |
| Zinc | 10 | mg | 5 | mg |
| Chromium | 40 | μg | 20 | μg |

ANTI-INFLAMMATORY

| | | | | |
|---|---|---|---|---|
| ALA (ω3) | 1000 | mg | 500 | mg |
| Curcumin | 240 | mg | 120 | mg |

Furthermore, the amino acids present in the composition (amino acids making up proteins and free amino acids) are the following:

| | Quantity for a daily | |
|---|---|---|
| | ration g | Quantity for a dose g |
| Leucine | 3.50 | 1.8 |
| Isoleucine | 1.90 | 0.9 |
| Valine | 2.20 | 1.1 |
| phenyl + tyrosine | 4.00 | 2.0 |
| Tryptophan | 1.05 | 0.5 |
| Neutral amino acids | 12.60 | 6.3 |
| Arginine | 3.60 | 1.8 |
| Threonine | 2.50 | 1.25 |

### EXAMPLE 5

The composition of example 5 is made up of:

| | |
|---|---|
| Quantity for a | Quantity for |
| daily ration | a dose |

PROTEINS

| | | | | |
|---|---|---|---|---|
| Peas | 12 | g | 6 | g |
| Rice | 8 | g | 4 | g |

FREE AMINO ACIDS

| | | | | |
|---|---|---|---|---|
| Leucine | 1.5 | g | 0.75 | g |
| Tryptophan | 0.5 | g | 0.25 | g |
| Taurine | 1.0 | g | 0.5 | g |
| Arginine | 0.5 | g | 0.25 | g |

MICRONUTRIENTS

| | | | | |
|---|---|---|---|---|
| Vitamin D | 5 | μg | 2.5 | μg |
| Vitamin B6 | 1.4 | mg | 0.7 | mg |
| Vitamin B9 | 200 | μg | 100 | μg |
| Vitamin E | 12 | mg | 6 | mg |
| Milk calcium | 600 | mg | 300 | mg |
| Marine | 300 | mg | 150 | mg |
| magnesium | | | | |
| Zinc | 10 | mg | 5 | mg |
| Chromium | 40 | μg | 20 | μg |

ANTI-INFLAMMATORY

| | | | | |
|---|---|---|---|---|
| ALA (ω3) | 1000 | mg | 500 | mg |
| Curcumin | 240 | mg | 120 | mg |

Furthermore, the amino acids present in the composition (amino acids making up proteins and free amino acids) are the following:

| | Quantity for a daily | |
|---|---|---|
| | ration g | Quantity for a dose g |
| Leucine | 4.2 | 2.1 |
| Isoleucine | 0.9 | 0.45 |
| Valine | 1.1 | 0.7 |
| phenyl + tyrosine | 2.0 | 1.0 |
| Tryptophan | 0.7 | 0.35 |
| Neutral amino acids | 8.9 | 4.45 |
| Taurine | 1.0 | 0.5 |
| Arginine | 2.2 | 1.1 |
| Threonine | 0.8 | 0.4 |

### EXAMPLE 6

The composition of example 6 is made up of:

| | |
|---|---|
| Quantity for a | Quantity for |
| daily ration | a dose |

PROTEINS

| | | | | |
|---|---|---|---|---|
| Peas | 24 | g | 12 | g |
| Rice | 16 | g | 8 | g |

FREE AMINO ACIDS

| | | | |
|---|---|---|---|
| Tryptophan | 0.25 | 0.12 | g |
| Taurine | 1.0 | 0.5 | g |

MICRONUTRIENTS

| | | | | |
|---|---|---|---|---|
| Vitamin D | 5 | μg | 2.5 | μg |
| Vitamin B6 | 1.4 | mg | 0.7 | mg |
| Vitamin B9 | 200 | μg | 100 | μg |
| Vitamin E | 12 | mg | 6 | mg |
| Milk calcium | 600 | mg | 300 | mg |
| Marine | 300 | mg | 150 | mg |
| magnesium | | | | |
| Zinc | 10 | mg | 5 | mg |
| Chromium | 40 | μg | 20 | μg |

ANTI-INFLAMMATORY

| | | | | |
|---|---|---|---|---|
| ALA (ω3) | 1000 | mg | 500 | mg |
| Curcumin | 240 | mg | 120 | mg |

Furthermore, the amino acids present in the composition (amino acids making up proteins and free amino acids) are the following:

| | Quantity for a daily | |
|---|---|---|
| | ration g | Quantity for a dose g |
| Leucine | 3.2 | 1.6 |
| Isoleucine | 0.9 | 0.45 |
| Valine | 1.1 | 0.6 |
| phenyl + tyrosine | 2.0 | 1.0 |
| Tryptophan | 0.7 | 0.35 |
| Neutral amino acids | 7.9 | 4.0 |
| Arginine | 3.4 | 1.7 |
| Taurine | 1.0 | 0.5 |
| Threonine | 1.5 | 0.7 |

### EXAMPLE 7

The composition of example 7 assumes the form of a powder containing 43 g of active molecules and excipients.

The active molecules are the following:

| | |
|---|---|
| Quantity for a | Quantity for |
| daily ration | a dose |

PROTEINS

| | | |
|---|---|---|
| Peas | 9 g | 4.5 g |
| Calcium | 7 g | 3.5 g |
| caseinate | | |

Furthermore, the amino acids present in the composition (amino acids making up proteins) are the following:

| | Quantity for a daily | |
|---|---|---|
| | ration g | Quantity for a dose g |
| Leucine | 1.43 | 0.72 |
| Isoleucine | 0.83 | 0.41 |
| Valine | 0.9 | 0.45 |
| phenylalanine + tyrosine | 1.65 | 0.82 |
| Tryptophan | 0.21 | 0.10 |
| Neutral amino acids | 5.0 | 2.5 |
| Glutamine | 3.40 | 1.7 |
| Arginine | 1.1 | 0.5 |
| Threonine | 0.65 | 0.3 |

### Evaluation of the Efficacy of the Composition According to the Invention

The composition according to the invention underwent a 12-week random, double-blind clinical trial in comparison to the product described in application FR2981544.

The study was carried out on patients:
with an excessive waist circumference relative to the IDF 2006 (80 cm for women and 94 cm for men)
with at least two cardiometabolic risk factors chosen from among: high blood pressure, high glycaemia, high triglycerides, low HDL cholesterol.

Patients followed a diet appropriate for their food habits, balanced (50% carbohydrates, 35% lipids, 15% proteins; carbohydrates with glycemic load below 10), hypocaloric (restriction of 600 kcal on calculated Resting Energy Expenditure [REE]) and including two doses per day of the composition according to the invention (that of the example 7) providing 380 kcal taken into account in the daily ration. A physical activity of at least 5000 steps (measured using a pedometer) was prescribed. This intensive phase was to be stopped after 12 weeks.

This intensive phase should be followed by a 4-week stabilization phase including a balanced diet with no calorie restriction and taking the composition according to the invention (that of example 7) per day.

When the results were analyzed, it was revealed that there was indeed a difference resulting in 2 distinct groups consisting of patients with more fat (greater visceral body fat mass, total fat mass and hypertriglyceridemia) and patients who had more of a hyperglycemic tendency; the former responded better to the composition of invention FR2981544, and the latter to the composition according to the invention.

A sub-group of 27 hyperglycemic subjects therefore yielded better results on all of the parameters.

The obtained results, given in the intent to treat population (i.e., 53 people for the entire group and 27 for the hyperglycemia subgroup) are provided in the table below:

| | TOTAL | | | |
|---|---|---|---|---|
| | SUB- | | | |
| | JECTS | | SUBJECTS | SUBJECTS |
| | treated | TOTAL | with | with |
| | by the | SUB- | HYPER- | HYPER- |
| | invention | JECTS | GLYCEMIA | GLYCEMIA |
| | BL | Δ% | invention BL | Δ% |
| POPULATION | 53 | | 27 | |
| MGV cm² | 181.6 | −8.4% | 198.6 | −9.3% |
| MGT cm² | 510.5 | −6.6% | 528.2 | −7.7% |
| MGSC cm² | 329.0 | −6.2% | 329.7 | −6.7% |
| MG DXA kg | 36.4 | −6.6% | 37.5 | −7.8% |
| MM DXA kg | 51.0 | −1.1% | 51.7 | −1.2% |
| TT cm | 103.4 | −3.5% | 104.3 | −4.3% |
| WEIGHT kg | 91.3 | −3.4% | 93.3 | −4.1% |
| TAS mm | 132.2 | −4% | 133.3 | −4% |
| TAD mm | 83.5 | −5.8% | 84.1 | −7% |
| G mmol | 5.6 | −4.7% | 6.1 | −7.6% (1) |
| INSULINEMIA | 12.5 | −10.1% | 11.5 | −15.7% |
| mUI/l | | | | |
| HOMA IR | 3.2 | −12.6% | 3.1 | −19.2% |
| HOMA B | 113.4 | −1.3% | 100.1 | −2.3% |
| HOMA S | 67.9 | +38.2% | 62.9 | +42.2% |
| CT mmol | 5.5 | −6.8% | 5.3 | −10.1% |
| HDL mmol | 1.2 | +11.4% | 1.2 | +20.8% |
| LDL mmol | 3.5 | −7.4% | 3.3 | −11.3% |
| TG mmol | 1.7 | −16.9% | 1.6 | −19% |
| PAI 1 ng/ml | 25.6 | −11.8 | 24 | −28% |
| MICROBIOTA | | | | |
| POPULATION | 27 | | 19 | |
| TOTAL FLORA | 73,838 | −1.2% | 74,344 | −1.9% |
| FIRMICUTES | 51,268 | −8.7% | 51,583 | −9.1% |
| BACTERIODETES | 15,063 | 65.8% | 14,581 | 66.9% |
| FIRMI/BACTER | 7.62 | −13.7% | 8.8 | −15.6% |

(1) average 5.52 mmol below the hyperglycemia threshold 5.60 mmol, therefore normalization.

It will in particular be noted that the composition according to the invention acts significantly (p<0.0001) on visceral fat with a loss of 8.4% for the entire population and 9.3% for the sub-group of hyperglycemic subjects. The total fat loss (p<0.0001) is 6.6% for the total population and 7.7% for the sub-group accompanied by a modest lean body mass loss of 1.1%, respectively.

It also acts on the risk factors and the fundamental factors of cardiometabolic risk:
Decrease glycaemia (p<0.0001) of 4.7% for the total population and 7.6% for the sub-group,
Decrease insulinemia (p=0.0046) of 10.1% for the total population and 15.7% for the sub-group,
Decrease HOMA IR (p=0.005) of 12.6% for the total population and 19.6% for the sub-group,
Decrease HOMA S (p=0.02) of 38.2% for the total population and 42.2% for the sub-group,
Decrease total cholesterol (p<0.0001) of 6.8% for the total population and 10.1% for the sub-group,
Increase HDL of 11.4% for the total population and 20.8% for the sub-group,
Decrease LDL (<0.0001) increase of 7.4% for the total population and 11.3% for the sub-group,
Decrease triglycerides (p<0.0001) of 16.9% for the total population and 19% for the sub-group,
Decrease PAI 1 (p=0.0075) of 11.8% for the total population and 28% for the sub-group,
In terms of microbiota, the fermicutes-to-bacteriodes ratio decreased by 13.7% for the total population and 15.6% for the subgroup.

It should be noted that the Per Protocol results, with 4 people deducted for noncompliance with the protocol, are even better.

The composition according to the invention therefore provides concrete and probative results on subjects with cardiometabolic risk, in particular those in the most dangerous position with the presence of hypertriglyceremia, which may be defined as a prediabetes situation with cardiovascular risk.

1. A composition formulated for oral administration in a human, the composition comprising at least one plant protein selected from the group consisting of pea proteins, rice proteins, and any mixture thereof, and at least one of the following:
at least one protein of animal origin selected from the group consisting of calcium caseinate, lactoserum proteins, and any mixture thereof;
at least one free amino acid selected from the group consisting of leucine, glutamine, isoleucine, valine, tryptophan, plant 5HTP, lysine, threonine, arginine and taurine, and any mixture thereof;
at least one micronutrient selected from the group consisting of vitamin D, vitamin B6, vitamin B9, vitamin E, milk calcium, marine magnesium, zinc, chromium, and any mixture thereof; and
at least one anti-inflammatory antioxidant compound of plant origin selected from the group consisting of ALA omega-3 fatty acid, purified EPA, protectin in PD1 form, protectin in PDX form, curcumin, and any mixture thereof.

2. The composition of claim 1, wherein the proteins of the composition are present in concentrate, isolate or hydrolysate form.

3. The composition of claim 1, wherein the protein(s) of the composition comprise at least the following amino acids: leucine, isoleucine, valine, glutamine, tryptophan, lysine, alanine, tyrosine, phenylalanine, and threonine.

4. The composition of claim 3, wherein leucine represents at least 7 wt % of all of the amino acids present in the proteins of the composition.

5. The composition of claim 3, wherein tryptophan, lysine and leucine together represent between 10 and 25 wt % of the amino acids present in the proteins of the composition.

6. The composition of claim 3, wherein branched amino acids and glutamine together represent between 20 and 40 wt % of the amino acids present in the proteins of the composition.

7. The composition of claim 3, wherein alanine and tryptophan together represent between 3 and 5 wt % of all of the amino acids present in the proteins of the composition.

8. The composition of claim 3, wherein tyrosine and phenylalanine together represent at least 7 and 10 wt % of all of the amino acids present in the proteins of the composition.

9. The composition of claim 1, wherein the composition comprises arginine and taurine in the weight ratio of arginine to taurine between 1.2 and 2.

10. The composition of claim 1, wherein the composition is formulated in a form selected from the group consisting of powder, granules, a ready-to-use beverage, a food bar, and an extruded form.

11. The composition of claim 10, wherein the composition weighs from 20 g to 60 g.

12. A method of preventing metabolic and cardiovascular pathologies in a patient with cardiometabolic risk, the method comprising administering to the patient an oral medical nutrition product comprising the composition of claim 1.

13. The method of claim 12, wherein hyperglycemia risk is the main cardiometabolic risk factor.

14. A method of treating a patient at risk of prediabetes, the method comprising administering to the patient an oral medical nutrition product comprising the composition of claim 1.

15. A method of treating a patient at risk of glycaemia, the method comprising administering to the patient an oral medical nutrition product comprising the composition of claim 1.

16. A method of treating a patient in the need of at least one of the following: decreasing waist circumference, normalizing blood pressure, increasing HDL cholesterol, decreasing LDL cholesterol, decreasing total cholesterol, decreasing triglycerides, decreasing visceral body fat mass, decreasing inflammation, and decreasing insulin resistance; the method comprising administering to the patient an oral medical nutrition product comprising the composition of claim 1.

17. A method for decreasing PAI 1 and insulinemia, and improving HOMA IR, HOMA B and HOMA S in a patient, the method comprising administering to the patient an oral medical nutrition product comprising the composition of claim 1.

18. A method of rebalancing microbiota species in a patient, the method comprising administering to the patient an oral medical nutrition product comprising the composition of claim 1 in the amount efficient for improving the firmicutes-to-bacteriodetes ratio.
